(19) United States
(12) Patent Application Publication
(10) Pub. No.: US 2006/0120513 A1
(43) Pub. Date: Jun. 8, 2006

(54) Adjustment device and adjustment method for a tomography apparatus

(76) Inventors: Thorsten Buttner, Kirchehrenbach (DE); Winfried Korber, Hallerndorf (DE); Hans-Jurgen Muller, Pretzfeld (DE); Elmar Rummert, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/261,991

(22) Filed: Oct. 27, 2005

(30) Foreign Application Priority Data
Oct. 27, 2004 (DE) 10 2004 052 281.2

(51) Int. Cl. A61B 6/08

(52) U.S. Cl. 378/206

(57) ABSTRACT

In an adjustment device and method for a tomography apparatus for aligning of a patient support device and an acquisition system relative to one another, a reference laser projects onto a reference element associated with the patient support device, the reference laser being directly associated with a rotary frame of the acquisition system, such that tolerances in the alignment between the reference laser and the acquisition system are prevented. Moreover, due to the arrangement of the reference laser directly on the rotary frame of the tomography apparatus, a simple calibration between laser beam and the acquisition plane of the acquisition system is ensured by a displacement of the reference laser in two different rotation angle positions which exhibit an angular interval therebetween of substantially 180 degrees.

### BACKGROUND OF THE INVENTION

1. Field of the Invention

The present invention concerns an adjustment device for a tomography apparatus for alignment of a patient support device and an acquisition system relative to one another, of the type wherein the acquisition system can rotate around a system axis on a rotary frame, and wherein the adjustment device has a reference element assigned to the patient support device and a reference laser projecting on the reference means. The invention also concerns a method for aligning a patient support device and an acquisition system relative to one another.

2. Description of the Prior Art

An adjustment device and an adjustment method of the above type are known for example, from United States Patent Publication No. 2004/0120467. For alignment of the patient support device relative to the acquisition system or the acquisition plane, a reference laser is positioned relative to the stationary part of the tomography apparatus such that a laser beam aligned perpendicular to the acquisition plane strikes a reference target.

The positioning of the reference laser in the adjustment position ensues in the known case by means of a pivotable frame borne on rollers or connected with the fixed part of the tomography apparatus.

Deviation between the projection of the laser beam and the reference means is initially determined for adjustment. The deviation specifies the displacement between the patient support device and the acquisition system. The displacement is subsequently either considered in the reconstruction of slice or volume images for a correct image representation, or is used for monitoring a manual alignment of the patient support device.

An important requirement for correct adjustment of the acquisition system relative to the patient support device is an initially precise arrangement of the reference laser relative to the acquisition system. If the laser beam generated by the reference laser is not situated perpendicularly to the acquisition plane of the acquisition system, a correct alignment between the patient support device and the acquisition system cannot be implemented.

### SUMMARY OF THE IVNENTION

An object of the present invention is to provide a tomography apparatus and a method for aligning a tomography apparatus of the above type wherein that a more precise but nevertheless simple alignment of a patient support device and an acquisition system relative to one another is ensured.

This object is achieved by an adjustment device and method for a tomography apparatus wherein a reference element is allocated to the patient support device and a reference laser projects onto the reference element for relatively aligning a patient support device and an acquisition system arranged on a rotary frame. According to the invention, the reference laser is directly associated with the rotary frame.

The reference laser and the acquisition system thus are directly mechanically coupled with one another via the rotary frame such that a precise alignment of the laser beam relative to the acquisition plane is possible within slight tolerances. Tolerances arising from an indirect connection between the reference laser and the acquisition system, as occur, for example, by arrangement of the reference laser on a pivotable or movable frame associated with the stationary part of the tomography apparatus, are prevented.

Due to the arrangement of the reference laser directly on the rotary frame, no mountings for positioning the reference laser that are complicated (in terms of design) to produce are required.

No interference with the operation of the tomography apparatus occurs with such an arrangement of the reference laser. The patient support device can be displaced in an unhindered manner for scanning an examination region.

The reference element is preferably a marking line that extends in the longitudinal direction of the patient support device. If the marking line has a length comparable to that of the patient support device, slight displacements from the position and perpendicular to the acquisition plane are quickly and definitively certainly visible.

In an embodiment of the invention, the reference laser generates a laser fan. A projection of the laser fan as a reference for the direction perpendicular to the acquisition plane enables a simple and fast monitoring of the position of the patient support device. For example, if the reference laser projects a laser fan onto a reference element in the form of a marking line, an offset of the position between patient support device and acquisition system can be optically detected in a reliable and simple manner by detecting deviation of the projection from the marking line.

The arrangement of the reference laser directly on the rotary frame has the further advantage that a simple and precise alignment between the reference laser and the acquisition plane of the acquisition system is possible. For this purpose, the reference laser can be displaced into two different rotation angle positions that exhibit an angular interval of substantially 180 degrees from one another. A first projection of the reference laser is detected in a projection plane at least one reference position in the direction of the system axis given a set first rotation angle position, and a second projection of the reference laser is detected in a projection plane given a set second rotation angle position. The displacement in different rotation angle positions of the reference laser ensues by an appropriate rotation of the rotary frame. The projections can be detected, for example, by a projection screen positioned at the respective reference position and oriented perpendicularly to the system axis.

At least one mounting bracket for mounting of a spirit level is provided on the rotary frame, such that the rotation angle positions of the reference laser can be very precisely adjusted.

A first distance (spacing) between the first and the second projection is preferably determined relative to a first reference position, and a second distance between the first and the second projection preferably is determined relative to a second reference position. Each distance is determined in a perpendicular direction relative to an imaginary connection line “reference laser position for the first rotation angle position—reference laser position for the second rotation angle position” and in a perpendicular direction relative to the system axis.

Two different displacement values of the patient support device relative to the acquisition system can be calculated independently of one another from the determined distances.

A first displacement value for alignment of the reference laser relative to the system axis can be determined from both distances, and on the other hand a second displacement value for alignment of the reference laser relative to the imaginary connection line “reference laser position for the first rotation angle position—reference laser position for the second rotation angle position” can be determined from both distances.

An alignment of the reference laser is possible dependent on the first displacement value and dependent on the second displacement value, such that the laser beam of the reference laser is aligned perpendicularly to the acquisition plane of the acquisition system.

A method for alignment of the patient support device and the acquisition system relative to one another includes the following steps:

displacement of the rotary frame such that the imaginary connection line “reference laser position—rotation center of the tomography apparatus” is aligned perpendicularly to the patient support plane of the patient support device,

determination of a deviation between a projection of the reference laser and in reference element,

displacement of the patient support device such that the projection of the reference laser is congruent with the reference element.

A method for calibration of the reference laser relative to the acquisition system includes the following steps:

displacement of the reference laser in two different rotation angle positions that exhibit an angular interval of substantially 180 degrees relative to one another, such that a first projection of the reference laser at a set first rotation angle position and a second projection of the reference laser at a set second rotation angle position can be detected in a projection plane at two reference positions differing in the direction of the system axis,

calculation of a first distance relative to the first reference position between the first and the second projection and of a second distance relative to the second reference position between the first and the second projection, each in a perpendicular direction relative to an imaginary connection line “reference laser position for the first rotation angle position—reference laser position for the second rotation angle position” in a perpendicular direction relative to the system axis,

determination of a first displacement value on the basis of both calculated distances, the first displacement value specifying the displacement of the reference laser relative to the system axis,

determination of a second displacement value on the basis of both calculated distances, the second displacement value specifying the displacement of the reference laser relative to the imaginary connection line “reference laser position for the first rotation angle position—reference laser position for the second rotation angle position”,

alignment of the reference laser dependent on the first and the second displacement values, such that the projection of the reference laser is in congruence with the system axis.

An alternative method version for calibration of the reference laser relative to the acquisition system includes the following steps:

displacement of the rotary frame in a first rotation angle position such that the imaginary connection line “reference laser position—rotation center of the tomography apparatus” is aligned perpendicularly to the patient support plane of the patient support device, such that a first projection of the reference laser in the form of a first projection line is detected at a reference position,

comparison with regard to parallelism of the first projection line with a vertical straight line (plumb line) aligned perpendicularly to the patient support plane of the patient support device,

alignment of the reference laser such that the first projection line is displaced in a second projection line that runs parallel to the vertical straight line,

displacement of the reference laser in a second rotation angle position that exhibits a displacement of substantially 180 degrees, such that a third projection line is detected,

determination of an interval center point between the second projection line and the third projection line proceeding parallel to one another,

alignment of the reference laser such that the second projection line and the third projection line proceed through the interval center point.

### DESCRIPTION OF OTHE DRAWINGS

FIG. 1 shows a tomography apparatus with an inventive adjustment device in a perspective view.

FIG. 2 shows the tomography apparatus according to FIG. 1, as seen from the front from the direction I indicated in FIG. 3, in a set first rotation angle position with the patient support device retracted for calibration of the adjustment device.

FIG. 3 shows the tomography apparatus in an axial longitudinal section according to section III-III in FIG. 2, with a laser beam that generates a projection at first and second reference positions being indicated for two rotation angle positions adjusted for calibration.

FIG. 4 shows the tomography apparatus in an axial longitudinal section according to section curve III-III in FIG. 2, wherein the first displacement angle is indicated.

FIG. 5 shows the tomography apparatus in the front view from direction II according to FIG. 3, wherein the second displacement angle is indicated.

FIG. 6 illustrates projections of a laser fan on a projection screen as are generated during a calibration event in accordance with the invention.

### DESCRIPTION OF THE PREFERRED EMBODIMENTS

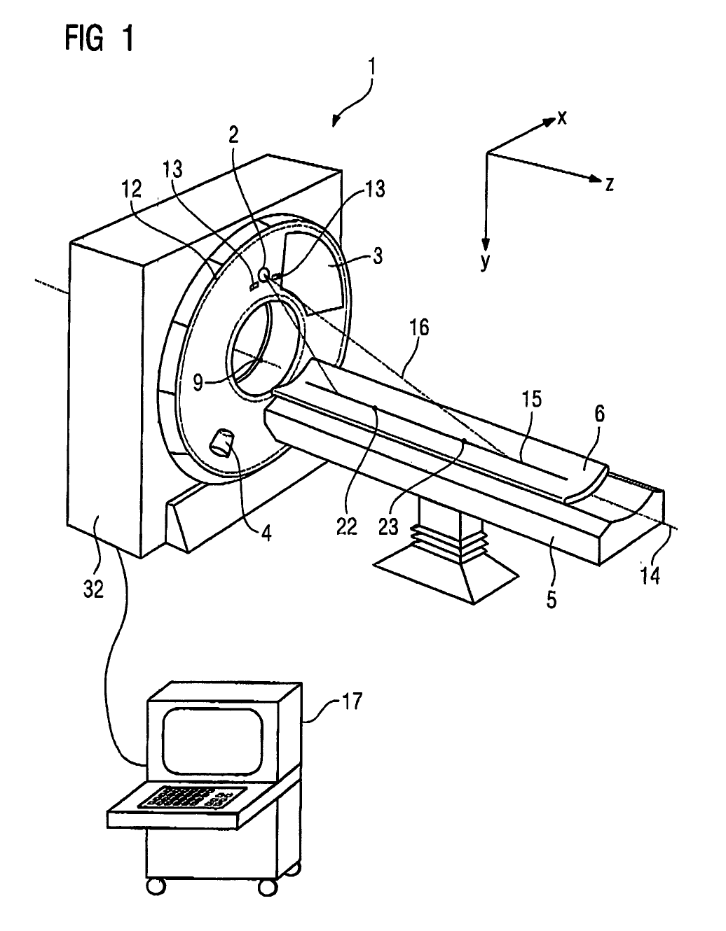

An inventive tomography apparatus (here in the form of a computed tomography apparatus 1) is shown in FIG. 1 in a perspective view. The computed tomography apparatus 1 has a patient support device, formed by a base 5 and a table plate 6, for supporting a subjects for example a patient, an acquisition system, formed by an x-ray radiation 4 and a radiation detector 3, arranged on a rotary frame 12, calculation unit (computer) 17 for reconstruction of slice and volume images and an adjustment device for mutual alignment of the patient support device and of the acquisition system.

The adjustment device is formed by two components, namely a reference laser 2 and a reference element 15. The reference laser 2 is arranged directly on the rotary frame 12 and thus is in direct mechanical connection with the acquisition system 3, 4. In this example, the reference laser 2 is arranged on the rotary frame 12 in addition to the radiation detector 3 of the acquisition system. In principle, arbitrary arrangements of the reference laser 2 on the rotary frame 12 are conceivable. The positioning will ensue in a practical manner from the point of view of a precise balancing of the rotary frame 12. The reference laser 2 can generate a laser beam in the form of a laser fan 16 and is aligned for adjustment such that the laser fan 16 projects onto the reference element 15. Recesses (not shown) or windows transparent for the laser fan 16 are appropriately provided in a housing 32 of the computed tomography apparatus 1, such that the laser fan 16 can project onto the reference element 15 without hindrance.

The reference element 15 is associated with the patient support device and, in the shown example, has the form of a marking line that extends in the longitudinal direction of the patient support device, in the z-direction of a Cartesian coordinate system shown in FIG. 1. The base 5 of the patient support device can be adjusted in terms of height and the table plate 6 is adjustable in the direction of the z-axis for supporting the subject. In the shown example, the marking line is directly on the surface of the table plate 6.

The x-ray radiator 4 of the acquisition system can be x-ray tube, and the detector 3, is composed of detector elements in columns and into rows in a detector array. The x-ray radiator 4 and the detector 3 are mounted opposite one another such that x-rays emanating from a focus of the x-ray radiator 4 penetrate through a measurement region and subsequently strike the detector 3.

In an operating mode of the computed tomography apparatus 1 for acquisition of slice and volume images of an examination region, a number of projections of the subject region to be examined are determined from different projection directions with continuous feed of the table plate 6 and simultaneous rotation of the acquisition system around the rotation center 9 of the computed tomography apparatus 1. The determined projections are transferred to the calculation unit 17 in the form of raw image data and are computed into slice or volume images according to a known reconstruction method. The result images can be displayed at a display unit integrated with the calculation unit 17.

For artifact-free acquisition of the raw image data, the longitudinal axis of the patient support device must be parallel to the feed direction and perpendicular to the acquisition plane, the acquisition plane being defined by the x-axis and the y-axis (shown in FIG. 1) of the Cartesian coordinate system.

The alignment of the patient support device relative to the acquisition system ensues in a simple and definitive manner by means of the inventive adjustment device. In the following embodiments, for better understanding, the rotation angle positions of the reference laser are specified in part as positions on a clock face. The alignment of the patient support device relative to the acquisition system includes the following steps:

displacement of the rotary frame 12 such that the imaginary connection line ‘reference laser position—rotation center of the tomography apparatus’ 11 is aligned perpendicularly to the supporting plane of the patient support device,

determination of a deviation between a projection of the reference laser 2 and the reference element 15,

displacement of the patient support device such that the projection of the reference laser 2 is congruent with the reference element 15.

The displacement of the reference laser 2 and of the patient support device in the position suitable for the alignment can be controlled by means of a program (provided for this purpose) executed in the calculation unit 17.

The shape of the projection can deviate from the laser fan 16. For example, projections in the form of a simple (linear) laser beam or a laser cross are possible. Naturally, it is not necessary that the reference element 15 exhibit the shape of a marking line. Other types of markings, for example reference points, can likewise be used.

The inventive adjustment achieves the following advantages:

Tolerances in the alignment between the reference laser 2 and the acquisition system are prevented by the direct arrangement of the reference laser 2 on the rotary frame 12.

No additional mountings for positioning of the reference laser 2 are required.

In terms of its operation for examination of a subject region, no interference in the operation of the computed tomography apparatus 1 is caused by the arrangement of the reference laser 2 on the rotary frame 12.

As to the direct arrangement of the reference laser 2 on the rotary frame 12 of the computer tomography apparatus 1, it is likewise possible in a certain and simple manner to align the reference laser 2 relative to the acquisition system or the acquisition plane.

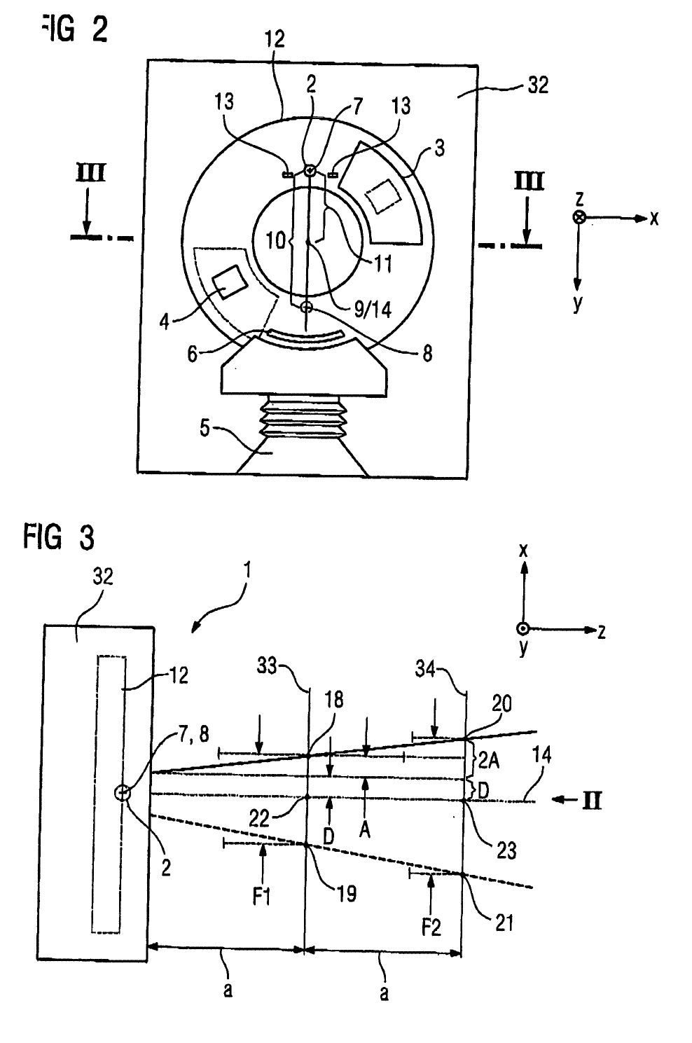

For calibration of the reference laser 2 relative to the acquisition system different reference positions 22, 23 in the direction of the system axis as well as, respectively, a first projection 18 and a second projection 19, and another first projection 20 and another second projection 21 (shown in FIG. 3) of the reference laser 2 are detected. The first projections 18 and 20 are acquired at a first rotation angle position 7 and the second projections 19, 21 are acquired at a second rotation angle position 8 of the reference laser 2, with the rotation angle positions 7 and 8 exhibit an angular separation of substantially 180 degrees from each other. In FIG. 2 the reference laser 2 of the computed tomography apparatus 1 is exemplarily shown in a set first rotation angle position 7 in a front view II according to FIG. 3. In this case, the first rotation angle position 7 corresponds to a 12 o'clock position and the second rotation angle position 8, which exhibits an offset of 180 degrees from the first rotation angle position, corresponds to a 6 o'clock position on an imaginary clock face. The second rotation angle position 8 is indicated in a dashed form in FIG. 2.

The first projections 18, 20 and the second projections 19 and 21 are detected at respective reference positions 22 and 23, for example by means of a suitably-positioned projection screen that is aligned perpendicularly to the system axis 14. So that the reference laser 2 can also radiate unhindered onto the projection screen in a set second rotation angle position 8, the patient support device is located in a sunken (retracted) position.

The projection image generated by the reference laser 2 corresponds to the projection screen of a projection line. The projection lines of the reference laser 2 in both rotation angle positions exhibit an offset dependent on the displacement of the reference laser 2 relative to the acquisition system. The first projections 18, 20 and the second projections 19 and 21 are respectively measured on the projection screen along a straight measurement lines 33 and 34 (as shown in FIG. 3) in a perpendicular direction relative to the imaginary connection line 10 (“reference laser position for the first rotation angle position—reference laser position for the second rotation angle position”) and in a direction perpendicular to the system axis 14. The position of the measurement in the direction of the connection line 10 can in principle be freely selected, but the position at which the straight measurement lines 33, 34 intersect the system axis 14 of the computed tomography apparatus 1 preferably is used.

In FIG. 3, the computed tomography apparatus 1 is shown in an axial longitudinal section according to section curve III-III in FIG. 2, with the first projections 18, 20 associated with the first rotation angle position 7 of the reference laser 2 and the second projections 19, 21 associated with the second rotation angle position 8 of the reference laser 2 being indicated. In comparison with the first reference position 22, in this example the second reference position 23 is arranged at twice as large a distance from the computer tomography apparatus 1. The first and second projections 18, 19, 20, 21 in this case respectively correspond to an intersection point that is formed from the straight measurement lines 33, 34 and the corresponding projection line of the reference laser 2.

A first distance F1 between the first and second projections 18, 19 is determined at the first reference position 22 and a second distance F2 from the first and second projections 20, 21 is determined at the second reference position 23. Two displacement values A, D for alignment of the reference laser 2 can be calculated from the two distances F1, F2 of the projections. The first displacement value A specifies the displacement of the reference laser 2 relative to the system axis 14 and the second displacement value D specifies the displacement of the reference laser 2 relative to the imaginary connection line ‘reference laser position of the first rotation angle position—reference laser position of the second rotation angle position’ 10. The first displacement valve and the second displacement value D can be determined, for example, by the following equations:
A=0.5·(F2−F1)
D=0.5·(2·F1−F2),
wherein F1 is the first distance, F2 is the second distance, A is the first displacement value and D is the second displacement value.

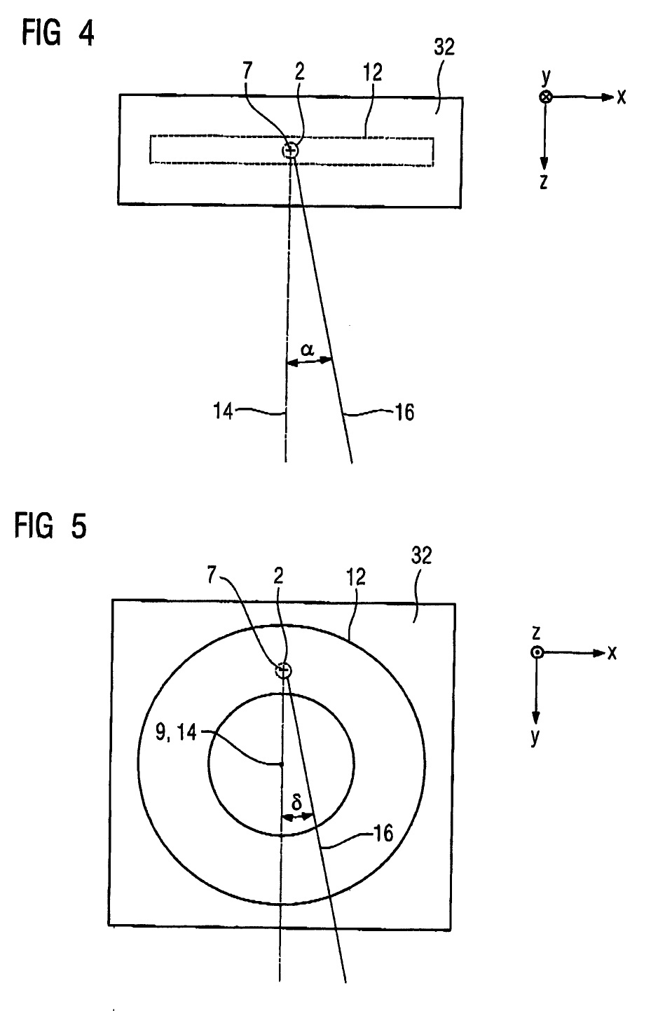

A first displacement angle α and a second displacement angle δ for correction of the alignment of the reference laser 2 relative to the acquisition system 3, 4 can be calculated form both displacement values A, D. The first displacement angle α specifies the displacement of the reference laser 2 relative to the system axis 14 of the computer tomography apparatus 1:
α=arc tan(A/a),
wherein α is the first displacement angle, A is the first displacement value and a is the distance of the first reference position 22 to the computer tomography apparatus 1. FIG. 4 shows the computed tomography apparatus 1 in an axial longitudinal section according to the section curve III-III in FIG. 2, wherein the first calculated displacement angle α is indicated.

The second displacement angle δ specifies the displacement of the reference laser 2 relative to the imaginary connection line 10:
δ=arc tan(D/R),
wherein δ is the second displacement angle, D is the second displacement value and R is the distance of the reference laser to the system axis of the computed tomography apparatus. FIG. 5 shows the computed tomography apparatus 1 in a front view II according to FIG. 3, wherein the second displacement angle δ is indicated.

The alignment of the reference laser 2 can also be corrected without the calculation of both displacement angles α, δ. In this case, the reference laser 2 is initially rotated around a laser axis parallel to the y-axis such that, relative to the second reference position 23, the laser fan 16 is displaced in the direction of the system axis 14 by twice the amount of the first displacement value A. The reference laser 2 is subsequently rotated around a laser axis parallel to the system axis 14 such that, relative to the second reference position 23, the laser fan is displaced in the direction of the system axis 14 by just the amount of the second displacement value D. As a result of the displacement, the laser fan 16 of the reference laser is congruent with the reference element 15 of the patient support device 5, 6.

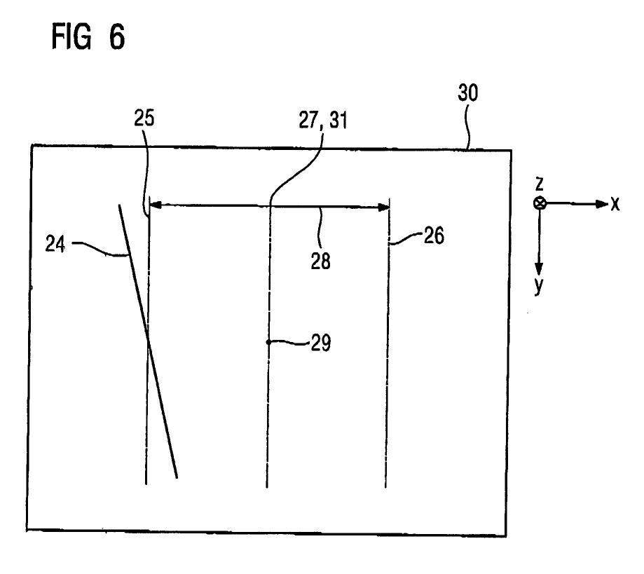

A calibration of the reference laser 2 relative to the acquisition system is, however, also possible with the projections of the reference laser 2 being evaluated at only one reference position, FIG. 6 as an example shows projections of the reference laser 2 that are generated in succession on a projection screen 30 at, for example, the first reference position 22 during such a calibration event. The reference laser 2 is displaced in a first rotation angle position 7 such that the imaginary connection line 11 (“reference laser position—rotation center of the computed tomography apparatus”) is aligned perpendicular to the patient support device. The first reference position 7 thus corresponds to a 12 o'clock position. In the first rotation angle position 7, the reference laser 2 generates a first projection in the form of a first projection line 24 which, with regard to parallelism, is compared with a straight vertical line 31 aligned perpendicularly to the supporting plane of the patient support device. The reference laser 2 is subsequently rotated around the system axis 14 of the computed tomography apparatus 1 until the first projection line 24 of the reference laser 2 coincides with a second projection line 25 that proceeds parallel to the straight vertical line 31.

The reference laser 2 is subsequently displaced in a second rotation angle position 8 that is at least essentially offset by 180 degrees from the first rotation angle position 7. The second rotation angle position 8 corresponds to the 6 o'clock position on a clock face. A third projection line 26 is detected in the second rotation angle position 8. The second and the third projection lines 25, 26 proceed parallel to one another and exhibit a certain spacing or distance 28 therebetween given a displacement from the y-axis of the laser fan 16 generated by the reference laser 2. The spacing center point 29 is determined from this distance 28. The reference laser 2 is now rotated around the certain connection line 11 such that the third projection line 26 is transferred in a fourth projection line 27 that proceeds through the spacing center point 29. It is naturally also possible to reset the reference laser 2 in the first rotation angle position 7 and to rotate the aforementioned imaginary connection line 10, such that the second projection line 25 proceeds through the interval center point 29.

Although modifications and changes may be suggested by those skilled in the art, it is the intention of the inventors to embody within the patent warranted hereon all changes and modifications as reasonably and properly come within the scope of their contribution to the art.

We claim as our invention:

1. In a tomography apparatus having an image data acquisition system rotatable around a system axis on a rotary frame, and a patient support device, the improvement of an adjustment device for aligning the acquisition system and the patient support device relative to each other, comprising:
a reference element allocated to said patient support device; and
a reference laser that emits a laser beam projecting onto said reference element, said reference laser being directly mounted on said rotary frame.

2. An adjustment device as claimed in claim 1 wherein said reference element comprises a marking line.

3. An adjustment device as claimed in claim 1 wherein said reference laser emits a laser fan as said laser beam.

4. An adjustment device as claimed in claim 1 comprising a horizontal lever indicator allowing adjustment of a rotation angle position of said reference laser, and a mounting bracket for said horizontal level indicator, in which said horizontal level indicator is received and held, on said rotary frame.

5. An adjustment device as claimed in claim 1 wherein said reference laser is mounted to said rotary frame allowing displacement of said reference laser, by rotation of said rotary frame, to two different rotation angle positions separated from each other by substantially 180°, a first of said rotation angle positions comprising a first reference position at which said laser emits a first projection at a set first rotation angle, and a second of said rotation angle positions comprising a second reference position at which said laser emits a second projection at a second rotation angle position detectable in a projection plane.

6. An adjustment device as claimed in claim 5 comprising a calculation unit connected to said adjustment device that determines a first distance between said first projection and said second projection relative to said first reference position, and a second distance between said first projection and said second projection relative to said second reference position, each in a direction perpendicular to an imaginary connection line, in a direction perpendicular to the system axis, between said reference laser at said first rotation angle position and said reference laser at said second rotation angle position.

7. An adjustment device as claimed in claim 6 wherein said calculation unit calculates a first displacement value from said first distance and said second distance, said first displacement value specifying displacement of said reference laser relative to said system axis.

8. An adjustment device as claimed in claim 7 wherein said calculation unit calculates a second displacement value from said first and second distances, said second displacement value specifying displacement of said reference laser relative to said imaginary connection line.

9. An adjustment device as claimed in claim 8 wherein said calculation unit controls alignment of said reference laser dependent on said first displacement value and said second displacement value to align said reference laser perpendicular to an image data acquisition plane of said acquisition system.

10. For a tomography apparatus comprising an image data acquisition system rotatable around a system axis on a rotary frame, a patient support device, a reference laser directly mounted on said rotary frame, and a reference element allocated to said patient support device, an adjustment method for aligning said patient support device and said acquisition system relative to each other, comprising the steps of:
displacing said rotary frame to cause an imaginary connection line between a position of said reference laser and a rotational center of said acquisition system to be aligned to a support plane of said patient supporting device;
electronically determining a deviation between a laser beam projection emitted by said reference laser and said reference element; and
automatically displacing said patient support device to make said laser beam projected by said reference laser congruent with said reference element.

11. A method as claimed in claim 10 comprising employing a marking line as said reference element.

12. A method as claimed in claim 10 comprising emitting a laser fan from said reference laser as said projection.

13. A method as claimed in claim 10 comprising aligning said reference laser relative to said acquisition system before aligning said patient support device and said acquisition system relative to each other, by an alignment procedure comprising the steps of:
by rotating said rotary frame, displacing said reference laser to two different rotation angle positions separated from each other by substantially 180°, and emitting a first projection from said reference laser at a first set rotation angle position and emitting a second projection from said reference laser at a set second rotation angle position, and detecting said projections at two reference positions different from each other in a direction of system axis:
electronically calculating a first distance relative to said first reference position between said first and second projections, and electronically calculating a second distance relative to said second reference position between said first and second projections, each in a direction perpendicular to an imaginary connection line between a position of said reference laser at said first angle position and a position of said reference laser at said second rotation angle position, in a direction perpendicular to the system axis;
electronically determining a first displacement value from said first and second distances, said first displacement value specifying displacement of said reference laser relative to said system axis;
electronically determining a second displacement value from said first and second distances, said second displacement value specifying displacement of said reference laser relative to said imaginary connection line; and
aligning said reference laser to said acquisition system dependent on said first and second displacement values to cause a projection from said reference laser to be congruent with said system axis.

14. A method as claimed in claim 10 comprising aligning said reference laser relative to said acquisition system before aligning said patient support device and said acquisition system relative to each other, by an alignment procedure comprising the steps of:
displacing said rotary frame to cause said reference laser to assume a first rotation angle position at which an imaginary connection line between the reference laser at said firs rotation angle position and a center of said acquisition system is perpendicular to a support plane of said patient supporting device, and causing a first projection from said reference laser comprising a first projection line to be detected at a reference position;
comparing parallelism between said first projection line and a vertical straight line oriented perpendicularly to said support plane of said patient supporting device;
aligning said reference laser to cause said first projection line to be displaced in a second projection line proceeding parallel to said vertical straight line;
displacing said rotary frame to displace said reference laser to a second rotation angle position separated substantially 180° from said first rotation angle position, to cause a third projection line from said laser to be detected:
electronically determining an interval center point between said second projection line and said third projection line parallel to each other; and
aligning said reference laser to cause one of said second projection line or said third projection line to proceed through said interval center point.
